(19) United States
(12) Patent Application Publication
(10) Pub. No.: US 2025/0049700 A1
(43) Pub. Date: Feb. 13, 2025

(54) Wound-Cleansing Method

(71) Applicant: Farrow Medtech LLC, College Station, TX (US)

(72) Inventor: Wade P. Farrow, Charlotte, NC (US)

(21) Appl. No.: 18/932,569

(22) Filed: Oct. 30, 2024

Related U.S. Application Data
(63) Continuation-in-part of application No. 18/140,414, filed on Apr. 27, 2023.
(60) Provisional application No. 63/547,140, filed on Nov. 2, 2023.
(60) Provisional application No. 63/336,661, filed on Apr. 29, 2022.

(51) Int. Cl. A61K 9/00; A61P 17/02

(52) U.S. Cl. CPC A61K 9/0014; A61P 17/02

(57) ABSTRACT

A method of treating a wound may include applying a staining agent to an area comprising a wound bed. After waiting to allow the staining agent to create a stained region on the area, a user may debride the stained region by placing a debridement device in contact with the stained region and moving the debridement device with respect to the stained region. This may remove at least a portion of a material forming the stained region. The staining agent may be methylthioninium chloride.

### CROSS-REFERENCE TO RELATED APPLICATION(S)

This application (1) claims the benefit of U.S. Provisional Patent Application Ser. No. 63/547,140 filed Nov. 2, 2023 and (2) is a continuation-in-part of U.S. patent application Ser. No. 18/140,414 filed Apr. 27, 2023, which claims the benefit of U.S. Provisional Patent Application Ser. No. 63/336,661 filed Apr. 29, 2022, each of which is hereby incorporated by reference.

### TECHNICAL FIELD

This disclosure relates to wound care, and more particularly, to novel systems and methods for cleansing, debriding, and/or desloughing wounds.

### BACKGROUND

Different types of devitalized tissue commonly appear in wound beds. Such tissue may interrupt granulation and delay healing. For example, devitalized tissue may stimulate the overproduction of matrix metalloproteases (MMPs) and thereby slow the healing process.

Additionally, devitalized tissue may provide an environment favorable to bacteria. Bacterial biofilms grow on 60-90% of chronic wounds and about 6% of acute wounds. Bacterial biofilms are low grade infections, cause chronic inflammation, impair wound healing, and increase risk of cellulitis.

Various technologies and/or methods have been developed to improve wound care. However, those technologies and/or methods have certain drawbacks. For example, ultrasonic debridement (e.g., debridement involving oscillations at about 20 kHz or above) is too expensive to be widely adopted, has a large spray pattern, and involves use of equipment that must be autoclaved between uses. Conversely, pulse lavage is often too untidy for use in clinic or emergency room settings.

Accordingly, what is needed are improved systems and methods for cleansing and/or desloughing wounds and removing bacterial biofilms. It would be an advance in the art to provide a cleansing pad specially designed for debridement and/or desloughing of wounds.

### SUMMARY

Disclosed herein are implementations of a wound-cleansing pad that may be used to clean and debride wounds and may be used in conjunction with other wound cleaning tools, and methods for production and use.

A wound-cleansing pad may be comprised primarily of a scrubbing element and a backing element. The scrubbing element may be connected to the backing element by one or more welds (e.g., ultrasonic or thermal welds). The welds may create a surface texture, or macro texture, on the top of the scrubbing element, which top side may be used to scrub and clean a wound.

A wound-cleansing pad may comprise a scrubbing element having a top side and a back side, wherein the scrubbing element comprises any suitable reticulated foam material. The wound-cleansing pad may also comprise a backing element having a front side and rear side, wherein the backing element comprises a polymeric film that provides a barrier layer, which blocks liquids and similar materials from passing through the back side of the scrubbing element. The wound-cleansing pad may also comprise a plurality of welds connecting the back side of the scrubbing element to the front side of the backing element, wherein the plurality of welds creates a surface texture on the top side of the scrubbing element.

In another implementation, a wound-cleansing pad may comprise two separate scrubbing elements. Each of the scrubbing elements may be attached to a single backing element. Each of the two scrubbing elements may have similar or different surface textures. Each of the two scrubbing elements may have similar or different porosities. Also, a wound-cleansing pad may comprise any reasonable number of scrubbing elements depending on the intended use of the wound-cleansing pad.

In another implementation, a wound-cleansing pad may further comprise a port, or valve. The port may be connected to the backing element in a manner that allows a gas to pass through the port and through the scrubbing element. This implementation may allow a gas to pass either way through the scrubbing element and port. For example, a sterilizing gas may be passed through the port and the scrubbing element onto a wound under the wound-cleansing pad, or a negative pressure may be applied to the wound through the port and the scrubbing element, thereby creating a vacuum over the wound that may help in the desloughing and debridement of the wound and/or stimulating granulation tissue formation.

A wound-cleansing pad may be used in a variety of ways. A wound-cleansing pad may be used with a gel, or cleansing gel, to help clean and debride a wound. Any suitable cleansing gel may be used. The selection of a cleansing gel may depend on the desired result after the scrubbing of the wound. A wound-cleansing pad with a port may be used in a way that allows a negative pressure to be applied to a wound.

Thus, there are a variety of implementations of a wound-cleansing pad and a variety of uses for each type of wound-cleansing pad. This allows for greater options in the treatment and care of various types of wounds.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a front plan view of one implementation of a wound-cleansing pad.

FIG. 2 is a rear plan view of the wound-cleansing pad of FIG. 1.

FIG. 3 is a side cross-sectional view of the wound-cleansing pad of FIG. 1.

FIG. 4 is a rear plan view of another implementation of a wound-cleansing pad.

FIG. 5 is a side cross-sectional view of the wound-cleansing pad of FIG. 4.

FIG. 6 provides a rear plan view of multiple alternative implementations of wound-cleansing pads.

FIG. 7A is a top view of one implementation of a wound-cleansing pad that may be used as a postoperative wound dressing with negative pressure.

FIG. 7B is a side view of one implementation of a wound-cleansing pad that may be used as a postoperative wound dressing with negative pressure.

FIG. 7C is a top view of an alternative implementation of a wound-cleansing pad that may be used as a postoperative wound dressing with negative pressure.

### DETAILED DESCRIPTION

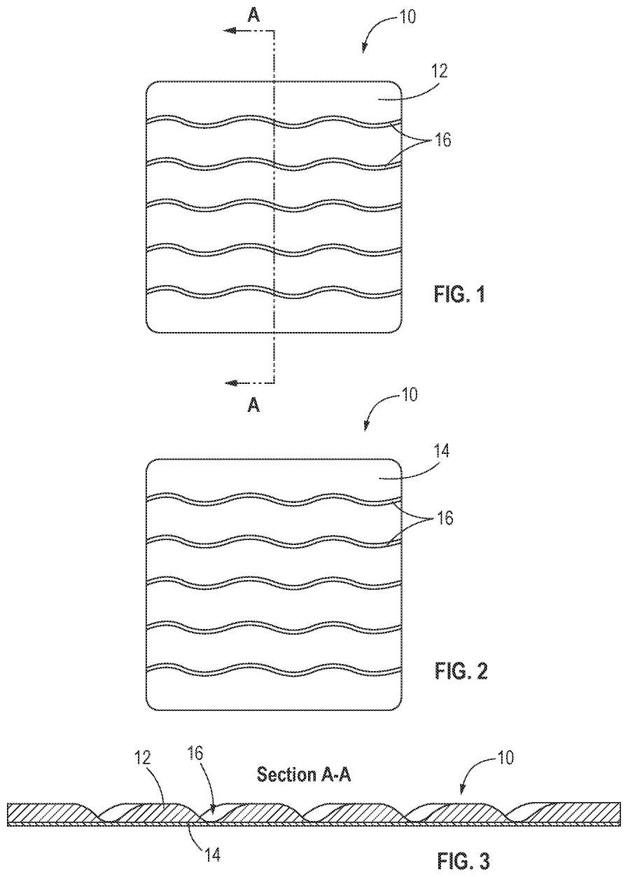

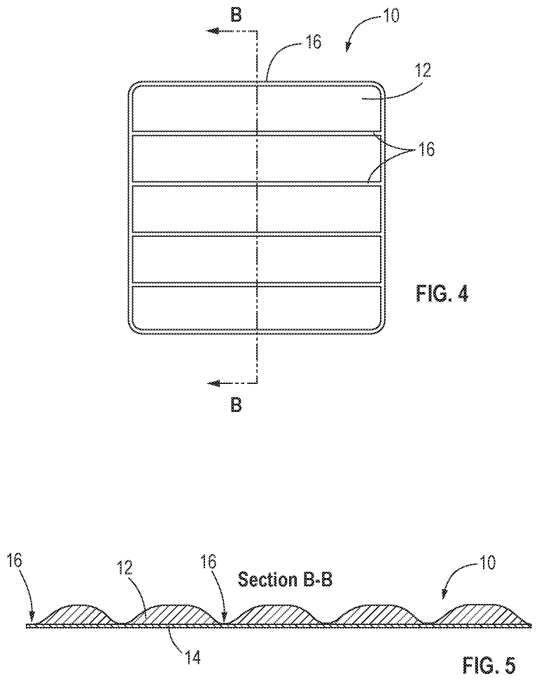

Referring to FIGS. 1-5, devitalized tissue appearing in a wound bed may include fibroslough (primarily fibrinous tissue), leukoslough (primarily white blood cell accumulation), necroslough (primarily necrotic tissue), and bioslough (primarily biofilm). Slough may be defined as devitalized tissue comprising primarily fibrin, but it may also include white blood cells, debris, and the like. The presence of slough may interrupt granulation and delay healing. It may also provide an environment in which bacteria may grow and spread. There is also an infection continuum in wounds, proceeding from contaminated wound bed (bacteria on surface of wound but not replicating), colonized (bacteria are living on wound bed and replicating), early biofilm formation, mature biofilm formation, and local to systemic infection with biofilm seeding. Accordingly, to promote healing, a wound-cleansing pad 10 may be used as a scrubbing tool or part of a scrubbing method to facilitate the removal of devitalized tissue from a wound. This removal may be described as wound cleansing and/or desloughing and/or debridement.

Depending on various factors, devitalized tissue in a wound bed may have different consistencies. For example, the consistency of devitalized tissue may be described as (1) mucinous (i.e., slimy and soft), (2) gelatinous, (3) stringy and/or clumpy, (4) fibrinous, and/or (5) leathery. Depending on the consistency of the devitalized tissue, different methods may be needed. For example, a tool and/or method suitable for debridement of devitalized tissue that is “leathery” may be too aggressive for devitalized tissue that is “mucinous,” or the like. Conversely, a tool and/or method suitable for cleansing and/or desloughing may be too soft to debride devitalized tissue that is fibrinous or leathery. Accordingly, a wound-cleansing pad 10 may be adaptable to provide proper cleansing and/or desloughing of devitalized tissue that is mucinous, gelatinous, stringy, fibrinous, and/or leathery.

Bacterial biofilms can be found in more than about 80% of chronic wounds. Biofilms usually contain multiple bacterial species. Biofilms can be found in multiple places, including within a wound bed (i.e., to a depth of about 60-70 microns below the surface of a wound), on a wound surface, in slough, in fluids on and around a wound bed, etc. and can migrate onto the wound dressings as well. Accordingly, a wound-cleansing pad 10 may be used as a scrubbing and/or abrasive tool to disrupt bacterial growth, prevent the formation of bacterial biofilms, and/or treat bacterial biofilms.

Wound care may be complicated by the fact that a wide variety of people with a wide variety of medical experience and/or training are tasked with providing such care. For example, at home, patients with little to no medical experience or training are often tasked with providing their own wound care (e.g., tasked with fitting and/or changing their own wound dressings). For patients with more serious wounds, wound care may be provided at their respective homes by ancillary healthcare staff having various levels of wound-care training. Some ancillary healthcare staff may have associate nursing degrees. Others have bachelor's degrees in nursing. Still others may be specialized wound, ostomy, and continence nurses (WOCN). However, regardless of such educational background, ancillary healthcare staff will have different comfort levels and skill sets with respect to cleansing wounds and conducting even basic wound cleansing and/or debridement.

In institutional, long-term care settings such as nursing homes and long-term acute care (LTAC) hospitals, there may be wound-care teams with more advanced training and an enhanced skill set due to their repeated and regular treating of complicated wounds. In the offices of physicians and surgeons, wounds often need cleansing or mild surgical debridement. Such treatment is often performed in an operating room. However, such use of an operating room is often unnecessary and increases the cost of treatment and may increase morbidity depending on choice of anesthesia.

Accordingly, to address the wide variety of people tasked with providing wound care, a wound-cleansing pad 10 may be easy to use. Moreover, it may be configured so as to limit the potential for misuse or harmful use. Accordingly, both an inexperienced patient and a highly trained medical professional may both use a wound-cleansing pad 10 to beneficial effect.

A wound-cleansing pad 10 may have any suitable shape and size. Suitable shapes may include rectangular, square, rectangular or square with rounded corners, circular, elliptical, triangular, or the like. Suitable sizes may include lengths and widths in a range from about 40 mm to about 100 mm and thicknesses in a range from about 3 mm to about 10 mm. For example, in certain implementations, there may be two different sizes of wound-cleansing pads: a wound-cleansing pad 10 of a large size may be a square with rounded corners that has a width/length of about 80 mm and a thickness of about 5 mm; and a wound-cleansing pad 10 of a smaller size may be square with rounded corners that has a width/length of about 50 mm and a thickness of about 5 mm.

In selected implementations, a wound-cleansing pad 10 may include a scrubbing element 12 and a backing element 14. A scrubbing element 12 may be or comprise relatively porous or reticulated foam. For example, a scrubbing element 12 may comprise a relatively thin sheet (e.g., a sheet having a thickness in a range from about 3 mm to about 10 mm) of open cell foam (e.g., reticulated foam formed of polyethylene, polyether, polyurethane, or the like) having a porosity in a range from about 15 pores-per-inch to about 40 pores-per-inch. In one implementation, a scrubbing element may be approximately 5 mm thick and have a porosity of 35 pores per inch, although any dimensions may be utilized. In other implementations, the scrubbing element may be about 4 mm to about 5 mm thick and have a porosity of about 20 pores-per-inch.

Different instances of wound-cleansing pads 10 may include scrubbing elements 12 of different porosity, stiffness, abrasiveness (e.g., via coatings applied to a porous foam structure or thickness of the foam pores/polyurethane type to increase abrasiveness), or the like or a combination or sub-combination thereof. In other implementations, a scrubbing element 12 may include an anti-microbial agent that may protect the wound-cleansing pad 10 from fungal and/or bacterial growth. A wound-cleansing pad 10 may comprise one or more scrubbing elements 12, where a separate material is used to provide separate and distinct scrubbing elements with a single instance of a wound-cleansing pad 10. For example, a wound-cleansing pad 10 may comprise a first instance of a scrubbing element 12 having a porosity of approximately 15 pores-per-inch and a second instance of a scrubbing element 12 having a porosity of approximately 40 pores-per-inch. Accordingly, assembling a wound-cleansing pad 10 with a scrubbing element 12 having particular characteristics may define or control how gently or aggressively the wound-cleansing pad 10 cleanses or desloughs a wound of a patient.

A backing element 14 may be or comprise a barrier layer. For example, a backing element 14 may be or comprise a polymeric film. In selected implementations, a backing element 14 may be a polyethylene terephthalate (PET) film that is about 0.01 mm (0.5 mil) to about 0.13 mm (5 mil) thick. In one implementation, a backing element 14 may be a polyethylene terephthalate (PET) film that is about 0.05 mm (2 mil) thick. In one implementation, a backing element 14 may have a thickness of between about 0.002 inches (0.05 mm) and about 0.01 inches (0.25 mm), and may comprise a polyester film. A backing element 14 may comprise any suitable material with any suitable dimensions. A backing element 14 may be secured to a scrubbing element 12 in any suitable manner. In selected implementations, a backing element 14 may be “quilted” using a heat press (i.e., thermal weld) or a weld 16 formed via application of ultrasonic energy. For example, a thermal press with a suitable configuration may be used to secure the scrubbing element 12 to the backing element 14, wherein the temperature may be about 500° F., the pressure applied may be about 500-1000 lbs., and the pressure may be applied for approximately 20-40 seconds or less.

A backing element 14 may impart a certain structural strength or a slight resistance to bending that may support a scrubbing element 12 during use of a corresponding instance of a wound-cleansing pad 10. Alternatively, or in addition thereto, a backing element 14 may block cleansing liquids, cleansing gels, or the like from passing through a scrubbing element 12 during use of a wound-cleansing pad 10. That is, cleansing liquids, cleansing gels, or the like may aid in cleansing or desloughing a wound. Without a backing element 14, such materials may easily pass through a scrubbing element 12 (e.g., a scrubbing element 12 formed of a relatively thin sheet of porous foam) and leave a working interface between the scrubbing element 12 and the wound without the benefit of those materials. Accordingly, a backing element 14 may prevent or block such unwanted migration of cleansing liquids, cleansing gels, or the like.

In selected implementations, welds 16 or other connection mechanisms or methods may create texture (e.g., macro texture) for a wound-cleansing pad 10. Such texture may have a larger scale and scope than the porosity (e.g., micro texture) of a scrubbing element 12. For example, welds 16 or other connection mechanisms or methods may create or be associated with indentations or compressions in a scrubbing element 12. A weld 16 may be described as forming a line, a curve, a dot, and/or the like on the surface of a scrubbing element 12. Accordingly, as a wound-cleansing pad 10 is moved back and forth across a wound bed, the overall or macro texture of the wound-cleansing pad 10 may facilitate better cleansing (e.g., create high and low pressure areas that facilitate cleansing and/or desloughing).

In certain implementations, a plurality of wound-cleansing pads 10 may be packaged and/or provided in a kit with a quantity of cleansing liquids, cleansing gels, or the like. In selected implementations, a dry cleansing agent or surfactant may be applied to a scrubbing element 12 in a manufacturing process. With the addition of a liquid (e.g., water, saline, mineral oil, or the like) to such a scrubbing element 12 at a time of use, the cleansing agent may hydrate and/or dissolve and facilitate or improve a wound-cleansing or wound-desloughing process. In certain implementations, cleansing liquids, cleansing gels, dry cleansing agents, antiseptic(s) or the like may include a surfactant that may compliment a scrubbing or cleansing action of a wound-cleansing pad 10 and assist in breaking down biofilms. In other implementations, a wound-cleansing pad 10 may include only the foam, the backing material, at least one surfactant, plus optionally an antiseptic, numbing medicine like lidocaine, and/or a biofilm staining agent. Suitable surfactants may include nonionic, ionic, or cationic surfactants. Suitable antiseptics may include polyhexanide (polyhexamethylene biguanide, PHMB), hypochlorous acid, methylene blue, silver ions, and iodine. In selected implementations, an antiseptic may be dried onto (e.g., powdered onto, applied via a solution where the solvent is later evaporated off) a wound-cleansing pad 10.

In certain implementations, six wound-cleansing pads 10 may be packaged and/or provided in a kit together with surfactant gel (e.g., surfactant in gel form) in a quantity sufficient to perform six wound cleansings. This may be a sufficient quantity of wound-cleansing pads 10 and surfactant to clean a wound three times per week for two weeks. Because biofilms can grow back in as little as 24-72 hours, use of wound-cleansing pads 10 and surfactant three times per week for two weeks (or use of two kits to enable one month of cleansing) may improve wound hygiene, allow better and repeated removal of a biofilm, and assist the patient to (over time) prevent the biofilm from returning due to the fact that inflammatory mediators reduce each time the biofilm is removed. The surfactant gel may be a 3-in-1 product (e.g., surfactant which may have antimicrobial properties plus numbing medicine) or a 4-in-1 product (e.g., surfactant which may have antimicrobial properties, plus numbing medicine, plus biofilm staining agent).

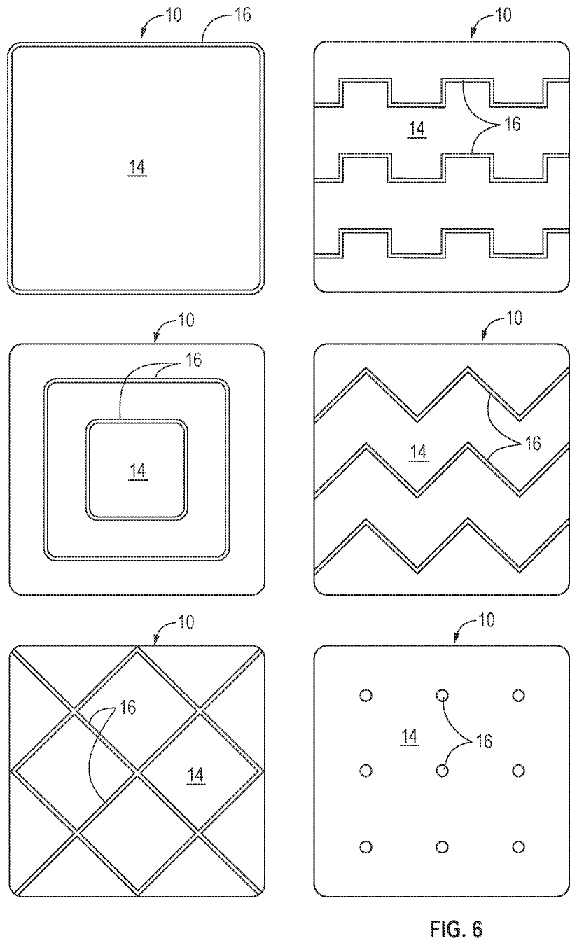

Referring to FIG. 6, welds 16 or other connection mechanisms or methods may have or form any suitable pattern or macro texture, or surface texture, on a wound-cleansing pad 10. In certain implementations, welds 16 or other connection mechanisms or methods may simply secure a perimeter of a scrubbing element 12 to a perimeter of a backing element 14. In other implementations, welds 16 or other connection mechanisms or methods may form lines, line segments, curves, or the like or combinations, sub-combinations, and/or intersections thereof to provide a desired macro texture on a wound-cleansing pad 10. In certain implementations, a greater number of welds 16 or other connection mechanisms or methods and/or the angles or pointed corners associated therewith may create a more aggressive macro texture. The macro texture, surface texture, or the like may be configured to have a wide variety of designs, including without limitation, parallel lines, parallel waves, concentric rectangles, concentric circles, latticed lines, and the like.

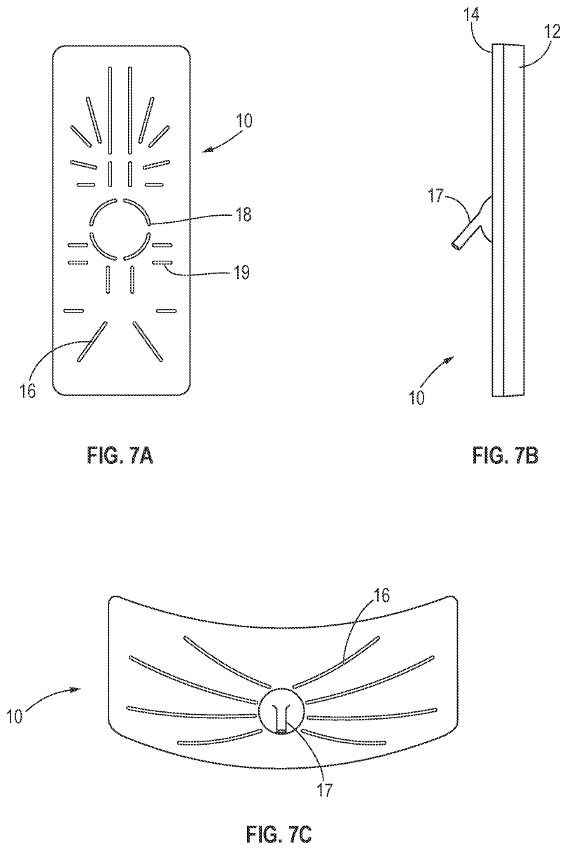

Referring to FIGS. 7A and 7B, in certain implementations or situations, a wound-cleansing pad 10 may function as a postoperative wound dressing with negative pressure. In these implementations, the welds 16 may be designed to apply or facilitate uniform and therapeutic suction across the wound-cleansing pad 10.

For example, FIG. 7A shows a wound-cleansing pad 10 suitable for use as a postoperative dressing for a knee or other joint. The welds 16 are designed to apply wider channels more distally, so that there is even suction applied across the incision. Additionally, other welds 18 are designed to maximize joint movement and patient comfort. For example, a wound-cleansing pad 10 suitable for use as a postoperative dressing may have welds that extend at least partially around the outside of the patella. Other welds 19 are designed to allow better flexion of the wound-cleansing pad 10.

A port 17 can be placed in a desirable location (e.g., more proximally or distally on the wound-cleansing pad 10). A port 17 may be of any suitable design or configuration and may be composed of any suitable material. Generally, a port 17 may be attached to a backing element 14 in a manner that allows a gas to pass through the scrubbing element 12 and exit the wound-cleansing pad 10 through the port 17. This port 17 may provide a connection to a negative pressure device directly or through tubing, thereby applying therapeutic suction to the wound bed and reducing the risk of postoperative wound dehiscence. Additionally, the wound-cleansing pad 10 may be designed such that suction may draw incision sides together, thereby contracting the entire dressing medially towards the middle on both sides and reducing tension on the incision and sutures. Reducing tension on suture lines may reduce the risk of wound dehiscence and pressure necrosis of sutures holding the incision together. Applying a negative pressure may also stimulate granulation tissue formation. The port 17 may be used for any suitable purpose. For example, the port 17 may be utilized to help treat a wound under the wound-cleansing pad 10 with a beneficial gas (i.e., a disinfectant, a germicidal, or a sterilizing gas).

FIG. 7C shows another implementation of a wound-cleansing pad 10 that may function as a postoperative wound dressing for use with negative pressure. In this implementation, the lines of the weld 16 are designed to enable the dressing to conform over an abdomen, such as a low transverse incision for a C-Section incision. These weld lines may also follow natural fold lines in the skin and/or apply uniform negative pressure to the entirety of the wound-cleansing pad 10.

In certain implementations, a surfactant (e.g., a surfactant in gel form) may be a nonionic surfactant. This may be advantageous as nonionic surfactant may be miscible with anionic, cat-ionic, and other nonionic surfactants. Examples of suitable nonionic surfactants may include PLURACARE block copolymers, which are synthetic copolymers of propylene oxide and ethylene oxide. Some suitable grades may include (but are not limited to) Pluragel F 68 (Poloxamer 188), F 108 (Poloxamer 338), F 127 (Poloxamer 407), Poloxamer 124, and Poloxamer 184. Other suitable surfactants may include PEG400, poloxamine 304, poloxamine 904, and poloxamine 908.

Pluronic grade surfactants may have gelation that is thermoreversible. In other words, the surfactant may have a gelatinous consistency when applied to a wound. However, after application and as the surfactant warms with body heat, it may become less gelatinous and more liquid like. This may facilitate application of the surfactant to the patient (e.g., facilitate the surfactant sticking to and not running off a wound surface), while also supporting diffusion of the surfactant across the wound surface and the removal of biofilm and other cellular debris.

In certain implementations, a surfactant gel may contain polyethylene glycol 400 (polymeric co-surfactant and solvent, which may form stable micelle clusters with Pluronic F127), mineral oil, Pluronic poloxamer F127, phenoxyethanol preservative, and tetrasodium EDTA. The pH may be adjusted for this surfactant gel by use of citric acid, as well as disodium phosphate as a buffering, sequestering agent and pH adjuster base. Because chronic wounds are typically at high pH, even a more neutral range pH of 6-7 may be acceptable, but an acidic gel may counteract the high pH in chronic wounds (e.g., a surfactant gel having a pH of about 4-5 may be preferred in certain implementations). Overall, a surfactant gel may have a pH in a range from about 4 to about 7. Higher acidity may also be achieved by adding an antiseptic such as small amounts of hypochlorite. In such implementations, the surfactant may better break up and isolate biofilm and bacteria, and the hypochlorite may function as a bactericide. The acidity may also assist in the debridement process by promoting reactions to help break up bonds in the wound bed, resulting in better debridement.

Osmolarity for a surfactant (e.g., a surfactant gel) may be neutral, or hyperosmolar to help collect and absorb drainage from the wound. Normal extracellular fluid has osmolarity of about 280 to about 295 mOsmol/kg. Osmolarity of a surfactant gel may be at least about 280 mOsmol/kg, and may be as much as 15% higher than extracellular fluid osmolarity (e.g., as high as about 340 mOsmol/kg). Thus, in certain implementations, a surfactant gel may have an osmolarity range of about 275 to about 340 mOsmol/kg. Nonionic surfactants may be hygroscopic and this may also assist in absorbing wound drainage and thus help trap and remove any inflammatory mediators, bacteria, biofilm, etc. that is broken up and isolated within the micelles.

Referring to Tables 1 and 2, in selected implementations, a surfactant gel may comprise a combination or sub-combination of trypsin, polyethylene glycol 400, mineral oil, Pluronic Poloxamer F127, phenoxyethanol, tetrasodium EDTA, and water. Trypsin dosage may be variable, depending on the age of the patient. Thus, different formulations may have different amounts of trypsin. In certain implementations, trypsin and/or one or more of the other ingredients may be omitted or have a similar product or ingredient substituted in its place.

TABLE 1

| Ingredient | Weight Percent |
|---|---|
| Trypsin | about 0.005% to about 0.15% |
| Polyethylene Glycol 400 | about 2.5% to about 7.5% |
| Mineral Oil | about 4.4% to about 13% |
| Pluronic Poloxamer F127 | about 10% to about 32% |
| Phenoxyethanol | about 0.5% to about 1.5% |
| Tetrasodium EDTA | about 0.05% to about 0.15% |
| Water | about 32% to about 96% |

TABLE 2

| Ingredient | Weight Percent |
|---|---|
| Trypsin | about 0.01% or omitted |
| Polyethylene Glycol 400 | about 5% |
| Mineral Oil | about 9% |
| Pluronic Poloxamer F127 | about 21% |
| Phenoxyethanol | about 1% |
| Tetrasodium EDTA | about 0.1% |
| Water | about 64% |

In selected implementations, a surfactant gel may be formed by (1) adding water to a main container, (2) adding tetrasodium EDTA to the main container, (3) mixing until all the solids are dissolved, (4) adding polyethylene glycol 400 to the main container and mixing until uniform, (5) adding trypsin to the main container and mixing until uniform, (6) adding mineral oil to the main container and mixing until uniform, (7) cooling the main container to about 5 degrees Celsius, (8) adding the Pluronic Poloxamer F127 and mixing until dissolved, (9) preparing 10% citric acid solution in water and/or a 10% disodium phosphate solution in water, (10) measuring the pH of the gel and adjust the pH to the desired value with either the citric acid or disodium phosphate solutions, (11) keeping the gel refrigerated until packaging, and the like or a sub-combination thereof.

In a surfactant gel, citric acid may be used as a pH adjuster acid. Disodium phosphate may be used as a buffering and sequestering agent and as a pH adjuster base. Polyethylene glycol 400 (e.g., Carbowax) may be a polymeric co-surfactant and solvent due to its ability to form stable micelle clusters with Pluronic F127. Polyethylene glycol 400 may also function at as wound sealing and healing agent, making it a potentially preferred candidate as a co-polymer. Mineral Oil may be used as an emolient, lubricant, therapeutic agent, and/or oleagenous oil that may assist in wound healing and/or play role in attracting bacteria onto itself. Pluronic Poloxamer F127 may thicken, emulsify, and/or gel the matrix. It may also function as a surfactant that prevents creation of biofilm. Phenoxyethanol may be a preservative that is safe for use on the skin and wounds. Tetrasodium EDTA may function as an anti-coagulant. Accordingly, if blood coagulation is desired, then tetrasodium EDTA may be removed in certain formulations (e.g., be replaced by a little more mineral oil).

In one implementation, a wound-cleansing gel may have a formulation as described in Table 3 below.

TABLE 3

| Ingredient | Weight Percent |
|---|---|
| 0.05% Hypochlorous Acid | about 64.2% |
| Saline solution | |
| Polyethylene Glycol 400 | about 5% |
| Mineral Oil | about 8.7% |
| Pluronic Poloxamer F127 | about 21% |
| Phenoxyethanol | about 1% |
| Tetrasodium EDTA | about 0.1% |

A wound-cleansing gel as described in Table 3 may be prepared in accordance with the following process. Add approximately 160.5 grams of distilled water into a 500 mL glass beaker and start mixing at 600 rpm in an overhead prop mixer. Transfer approximately 0.25 grams of Tetrasodium EDTA to the main beaker while mixing and mix until completely dissolved. Add approximately 2.5 grams of phenoxyethanol to the main beaker and mix until completely dissolved. Add approximately 12.5 grams of polyethylene glycol 400 to the main beaker and mix until completely dissolved. Add approximately 21.54 grams of mineral oil to the main beaker and mix until uniform. Transfer the batch into a freezer and cool to 4° C. while stirring on a magnetic stir plate with a steel bar. Monitor the temperature by a digital thermometer with a probe that is placed inside the batch. Remove the batch from the freezer and place it into a large container with ice to maintain the cold temperature while mixing using the prop mixer at 1200 rpm. Add approximately 52.5 grams of Poloxamer F127 to the batch slowly, making sure the temperature of the batch stays between 4° C.-5° C., and mix for at least 60 minutes at 1500 rpm. Prepare a 10% citric acid solution with distilled water. Add the citric acid solution to the batch dropwise while mixing at 1500 rpm until reaching the desired pH of 6.0. Package the batch while the temperature is between 4° C.-5° C. This process may be used to prepare approximately 250 grams of the wound-cleansing gel. This process may be scaled to produce larger quantities of the wound-cleansing gel.

In one implementation, a wound-cleansing gel may have a formulation as described in Table 4 below.

TABLE 4

| Ingredient | Weight Percent |
|---|---|
| 0.05% Hypochlorous Acid | about 64.2% |
| Saline solution | |
| Polyethylene Glycol 400 | about 5% |
| Mineral Oil | about 5.8% |
| Pluronic Poloxamer F127 | about 21% |
| Phenoxyethanol | about 1% |
| Lidocaine HCl | about 3% |

A wound-cleansing gel as described in Table 4 may be prepared in accordance with the following process. Add approximately 160.5 grams of distilled water into a 500 mL glass beaker and start mixing at 600 rpm in an overhead prop mixer. Add approximately 7.5 grams of Lidocaine HCl and mix until completely dissolved. Add approximately 2.5 grams of phenoxyethanol to the main beaker and mix until completely dissolved. Add approximately 12.5 grams of polyethylene glycol 400 to the main beaker and mix until completely dissolved. Add approximately 14.5 grams of mineral oil to the main beaker and mix until uniform. Transfer the batch into a freezer and cool to 4° C. while stirring on a magnetic stir plate with a steel bar.

Monitor the temperature by a digital thermometer with a probe that is placed inside the batch. Remove the batch from the freezer and place it into a large container with ice to maintain the cold temperature while mixing using the prop mixer at 1200 rpm. Add approximately 52.5 grams of Poloxamer F127 to the batch slowly, making sure the temperature of the batch stays between 4° C.-5° C., and mix for at least 60 minutes at 1500 rpm. Prepare a 10% citric acid solution with distilled water. Add the citric acid solution to the batch dropwise while mixing at 1500 rpm until reaching the desired pH of 6.0. Package the batch while the temperature is between 4° C.-5° C. This process may be used to prepare approximately 250 grams of the wound-cleansing gel. This process may be scaled to produce larger quantities of the wound-cleansing gel.

A wound-cleansing pad may be used in a variety of ways. In one implementation, a method for cleansing a wound may comprise, providing a wound-cleansing pad, wherein the wound-cleansing pad comprises a scrubbing element, a backing element, and a plurality of welds, wherein the scrubbing element is a reticulated foam, the backing element is a polymeric film that provides a barrier layer, and the plurality of welds forms a surface texture on the top of the scrubbing element. The method may further comprise providing a gel, wherein the gel may comprise a hypochlorous acid saline solution and a surfactant, or the gel may have any suitable formulation. The method may further comprise applying the gel, whichever gel is selected, to the wound-cleansing pad. The method may further comprise cleansing the wound by applying the wound-cleansing pad with the gel to the wound and removing slough from the wound, which may be accomplished by rubbing or scrubbing the wound with the wound-cleansing pad in a manner suitable for desloughing the wound, and/or debridement of the wound. Such a method may be utilized to cleanse a wide variety of wounds, including without limitation, wounds that may be described as mucinous, gelatinous, stringy, fibrinous, and leathery, or the like. Moreover, a desired porosity may be selected for the scrubbing element depending on the type of wound being cleansed. For example, a scrubbing element may have a porosity within the approximate range of 15 pores/inch to 40 pores/inch.

In another implementation, a method for cleansing a wound may comprise, providing a wound-cleansing pad, wherein the wound-cleansing pad comprises a scrubbing element, a backing element, a plurality of welds, and a port, wherein the scrubbing element is a reticulated foam, the backing element is a polymeric film that provides a barrier layer, the plurality of welds forms a surface texture on the top of the scrubbing element, and the port is connected to the backing element in a manner that allows a gas to be passed through the port and through the scrubbing element. The method may further comprise positioning the wound-cleansing pad on a wound and applying a negative pressure through the port to the wound. The method may further comprise positioning the wound-cleansing pad on a wound and applying a gas through the port onto the wound.

In another implementation, a method for cleansing a wound may comprise, providing a wound-cleansing pad, wherein the wound-cleansing pad comprises a scrubbing element, a backing element, a plurality of welds, and a port, wherein the scrubbing element is a reticulated foam, the backing element is a polymeric film that provides a barrier layer, the plurality of welds forms a surface texture on the top of the scrubbing element, and the port is connected to the backing element in a manner that allows a gas to be passed through the port and through the scrubbing element. The method may further comprise providing a gel, wherein the gel may comprise a hypochlorous acid saline solution, and/or citric acid solution, and/or hyaluronic acid solution and at least one surfactant, or the gel may have any suitable formulation. The method may further comprise applying the gel, whichever gel is selected, to the wound-cleansing pad. The method may further comprise cleansing the wound by applying the wound-cleansing pad with the gel to the wound and removing slough from the wound, which may be accomplished by rubbing or scrubbing the wound with the wound-cleansing pad in a manner suitable for desloughing the wound, and/or debridement of the wound. The method may further comprise positioning the wound-cleansing pad on a wound and applying a negative pressure through the port to the wound.

In selected implementations, methylthioninium chloride (hereinafter “methylene blue”) or some other staining agent (e.g., crystal violet or gentian violet, also known as methyl violet 10B or hexamethyl pararosaniline chloride) may be used in cleansing or debriding methods disclosed herein. For example, methylene blue may be applied to a wound bed and the surrounding tissues (e.g., the periwound skin). The methylene blue may stain certain elements or tissues (e.g., preferentially stain certain elements or tissues). For example, the methylene blue may stain biofilm, devitalized tissue, and the like. Accordingly, while the methylene blue may rinse off certain portions of the wound site (i.e., does not stain muscle, tendon, nerve, bone, intact skin, or fat), it does stain biofilm, devitalized tissue, and cartilage. This may aid a user in detecting the presence of the biofilm (which cannot be seen with the naked eye), seeing the extent of the biofilm, seeing where cleansing and/or debridement is needed, and/or seeing how effective their cleansing and/or debridement has been in removing the biofilm as well as devitalized tissue (which will be stained as well) and needs to be removed. In certain situations, cartilage may also be stained by methylene blue. Accordingly, a user may distinguish between stained biofilm and devitalized tissue and stained cartilage (if present) when cleansing or debriding a wound bed and surrounding tissues.

In certain implementations, methylene blue may be applied as part of a liquid, gel, or the like to an area comprising a wound bed and surrounding tissues (e.g., about a one-inch border of periwound skin). For example, the liquid, gel, or the like may comprise about 0.01 to about 0.5 weight percent of methylene blue. Alternatively, the liquid, gel, or the like may comprise about 0.01 to about 0.1 weight percent of methylene blue.

In selected implementations, methylene blue may be added to one or more of the formulations disclosed above. For example, methylene blue may be added in the concentrations indicated above to a formulation comprising hypochlorous acid. In certain implementations, methylene blue may be added in the concentrations indicated above to a formulation comprising lidocaine jelly or the like. In other implementations, methylene blue may be mixed with a saline solution. Accordingly, methylene blue may be added (e.g., added in the concentrations disclosed above) to any of the formulations disclosed in Tables 1-4 provided above.

In still other implementations, one or more individual packets of methylene blue (e.g., finely ground methylene blue) powder may be supplied alone or as part of a kit (e.g., a kit comprising a container of saline, a wound-cleansing pad as disclosed above, or the like or a combination or sub-combination thereof). Accordingly, a user may apply (e.g., sprinkle) the powder directly to a wetted wound bed and surrounding tissues, pour the powder into the container of saline before applying the saline to the wound bed and surrounding tissues, or the like to stain the biofilm and/or devitalized tissue.

After applying the methylene blue to the area (e.g., the wound bed and, optionally, certain surrounding tissues), the user may leave it in place to grant sufficient time for it to create a stained region (e.g., a region of material comprising stained biofilm and/or stained devitalized tissue) on the area. In certain implementations, this may comprise applying the methylene blue solution, gel, or the like to the wound bed and surrounding tissues and waiting about 3 to about 7 minutes. In other implementations, it may comprise applying the methylene blue solution, gel, or the like and waiting about 5 minutes. In selected implementations, applying a methylene blue solution, gel, or the like may comprise wetting or soaking gauze or the like with the solution, gel, etc. and placing the wetted gauze on the wound for a sufficient period (e.g., about 3 to about 7 minutes, about 5 minutes, or the like).

After application and waiting a sufficient period, the methylene blue solution, gel, or the like may be rinsed off, thereby revealing the stained region (e.g., material comprising stained biofilm, stained devitalized tissue, or the like). Accordingly, the user may see where to clean, debride, etc. Additionally, the staining may enable the user to accurately determine when the biofilm and/or devitalized tissue has been removed by the user's cleaning, debriding, or other efforts. That is, when the stained region (e.g., the material forming the stained region) is removed, it is an indicator that the biofilm and/or the devitalized tissue has been removed. Alternatively, after cleaning and/or debriding a wound bed and surrounding tissues, a user may apply (may again apply) the methylene blue solution, gel, or the like to determine whether all or a sufficient portion of the biofilm and/or devitalized tissue has been removed. Thus, the process of staining and cleansing/debriding may be iterated or repeated as necessary or desired (e.g., until no biofilm and/or devitalized tissue remains).

In certain situations, cleansing and/or debridement after staining may reveal increased or persistent stained areas in otherwise viable tissue. This may be an indication of active tissue infection. This may make it readily apparent that antibiotic treatment is likely necessary to irradicate the biofilm in otherwise viable tissue.

As set forth above, a wound-cleansing pad may be a debridement device used to contact the area (e.g., the wound bed and, optionally, certain surrounding tissues) and, while moving with respect to the area, remove biofilm and/or devitalized tissue that has been stained by methylene blue. Alternatively, other devices or tools (e.g., mechanized or powered tools) may be used to contact the area and, while moving with respect to the area, remove biofilm and/or devitalized tissue that has been stained by methylene blue. For example, one or more of the debridement devices, tools, or systems disclosed in U.S. patent application Ser. No. 18/074,475 filed Dec. 3, 2022 and entitled Wound-care Apparatus and Method for Cleansing, Desloughing, and Debriding Wounds, which is hereby incorporated by reference, may be used to remove biofilm and/or devitalized tissue that has been stained by methylene blue as set forth above or by some other method. Such tools may speed or produce a better therapeutic and/or analgesic effect. Moreover, when used in combination with methylene blue solution, gel, or the like, a user of one or more such tools may better gauge the effectiveness of the treatment. Thus, the user may ensure adequate cleansing and/or debridement without risking or moving into excessive or unnecessary disturbance of the wound bed and surrounding tissues. This may be particularly helpful for users (e.g., medical professionals, patients themselves, caregivers, or the like) who have less experience with wound cleansing and debridement.

In certain implementations, methylene blue may be added to a surfactant gel, so that it stains the biofilm and/or devitalized tissue, and application of a mechanized or powered tool can help break up and lift the biofilm and/or devitalized tissue off the wound bed and surrounding tissues. Hence, the method of using methylene blue, a surfactant gel, and a mechanized or powered tool (e.g., a handheld cleansing or debridement device) may create a point-of-care diagnostic and treatment system that is quick and easy to use, yet effective. Thus, diagnosis and treatment need not be delayed while samples are taken and tests are run. Rather, diagnosis and treatment can occur immediately at the point-of-care as the clinical situation dictates, thereby, improving patient care, reducing costs, reducing morbidity, and improving the speed and accuracy of clinical decision making.

While selected implementations disclosed above involve methylene blue, other staining agents (e.g., crystal violet) may be used in place of or in addition to methylene blue as desired or necessary. The concentration of such alternative staining agents may be selected to provide a desired visibility and may, therefore, be the same or differ from the concentrations disclosed above for methylene blue.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method of treating a wound, the method comprising:
applying a staining agent to an area comprising a wound bed;
waiting to allow the staining agent to create a stained region on the area;
debriding the stained region by placing a debridement device in contact with the stained region and moving the debridement device with respect to the stained region; and
removing, as a result of the debriding, at least a portion of a material forming the stained region.

2. The method of claim 1, wherein the material comprises at least one of biofilm or devitalized tissue stained by the staining agent.

3. The method of claim 2, further comprising revealing the stained region by rinsing off the area after the waiting.

4. The method of claim 2, wherein the staining agent is methylthioninium chloride.

5. The method of claim 1, wherein:
the staining agent is contained within a liquid or a gel; and
the applying the staining agent comprises applying the liquid or the gel to the area.
